(19) United States
(12) Patent Application Publication
(10) Pub. No.: US 2025/0339050 A1
(43) Pub. Date: Nov. 6, 2025

(54) SYSTEMS AND METHODS FOR ELECTROPHYSIOLOGICAL SIGNAL RECORDING AND POSITION OR MOTION MONITORING DURING MAGNETIC RESONANCE IMAGING

(71) Applicant: The General Hospital Corporation, Boston, MA (US)

(72) Inventor: Giorgio Bonmassar, Charlestown, MA (US)

(21) Appl. No.: 18/862,293

(22) PCT Filed: May 2, 2023
(86) PCT No.: PCT/US2023/066485

§ 371 Date: Nov. 1, 2024

Related U.S. Application Data
(60) Provisional application No. 63/363,992, filed on May 2, 2022.

(51) Int. Cl. A61B 5/055; A61B 5/06; A61B 5/11; A61B 5/291

(52) U.S. Cl. CPC A61B 5/055; A61B 5/062; A61B 5/11; A61B 5/291

(57) ABSTRACT

Systems and methods are provided that include acquiring, at a plurality of different times, an induced voltage within a plurality of coils arranged about the subject's head and positioned within a variable magnetic field. The method also includes acquiring electroencephalogram (EEG) data from a plurality of EEG sensors, wherein each EEG sensor is paired with a respective one of the plurality of coils. The method also includes determining, for each of the plurality of times, a position of each coil in the plurality of coils positioned in the variable magnetic field utilizing the induced voltage at the particular time and using the position of each coil in the plurality of coils to correlate the EEG data with at least one of an anatomical image or a functional image of the head of the subject.

This application is based on, claims priority to, and incorporates herein by reference for all purposes, U.S. Provisional Application Ser. No. 63/363,992, filed May 2, 2022.

### BACKGROUND

Electrophysiological brain signals are typically recorded by an electroencephalogram (“EEG”) system. The EEG system may be a device that measures the electrical activity in the brain via a multitude of electrodes attached to a patient's scalp by way of a cap or a special glue or paste and connected to the EEG system through wires called leads. The electrodes detect the electrophysiological signals, and the EEG system amplifies and records them onto paper or a computer for analysis by medical personnel.

Recording the EEG signals allows medical personnel to view information (e.g., a graph) reflecting the activity of billions of neurons in the brain. The pattern of activity in the recorded EEG signals or brain waves changes with the level of the patient's arousal—if the patient is relaxed, the graph shows many slow, low-frequency brain waves; if the patient is excited, the graph shows many fast, high-frequency brain waves.

While EEG provides useful temporal information regarding the brain's electrical activity, EEG provides very low spatial resolution and cannot be used for determining the exact location of the recorded activity in the brain. However, high spatial resolution is often important for diagnosing and treating many brain-related conditions such as epilepsy or seizures.

Magnetic resonance imaging (“MRI”) is able to provide high anatomical special resolution. Furthermore, functional MRI (“fMRI”) can be performed using an MRI system to acquire information about the function of the brain. MRI is a technique that utilizes magnetic and radio frequency (“RF”) fields to provide high-quality image slices of the brain along with detailed metabolic and anatomical information. Radio waves 10,000-30,000 times stronger than the earth's magnetic field are transmitted through the patient's body. This affects the patient's hydrogen atoms, forcing the nuclei into a different position. As the nuclei move back into place, they send out their own radio waves. An MRI scanner picks up those radio waves, and a computer converts them into images based on the location and strength of the incoming waves.

fMRI uses an MRI system to detect changes in cerebral blood volume, flow, and oxygenation that locally occur in association with an increased neuronal activity that may be induced by functional paradigms. This physiological response is often referred to as the “hemodynamic response.” The hemodynamic response to neuronal activity provides a mechanism for image contrast commonly referred to as the blood-oxygen level-dependent (BOLD) signal contrast. An MRI system can be used to acquire signals from the brain over a period of time. As the brain performs a task, these signals are modulated synchronously with task performance to reveal which brain regions are involved in completing the task. The series of fMRI-course images must be acquired at a high enough rate to see the changes in brain activity induced by the functional paradigm. In addition, because the neuronal activity may occur at widely dispersed locations in the brain, a relatively large 3D volume or multi-slice volume must be acquired in each time frame.

In order to take advantage of the high temporal resolution of EEGs and the high spatial resolution of MRIs and fMRIs, medical personnel have been seeking ways to simultaneously acquire EEG data and MRI or fMRI data. Such simultaneous recording would provide the high spatio-temporal resolution needed to study brain activity during different tasks, such as visual, auditory, or motor tasks. Currently, no single brain imaging technology can provide the resolution needed to study this brain activity. A combination of EEGs and MRIs/fMRIs would provide the required resolution while improving the accuracy of diagnosing many brain-related conditions.

The combination of EEG and MRI/fMRI is impractical or, at best, limited for a variety of fundamental reasons. First, the magnetic fields required for MRI require that no ferromagnetic materials be utilized near the MRI system. Beyond this real challenge with material choice and sensor design, the static, the changing magnetic and RF fields of an MRI/fMRI system can introduce significant undesirable artifacts into the EEG recordings. When EEG leads are placed inside an MRI scanner, even if dangerous currents are avoided, the rapidly changing RF fields may introduce signals that can obscure the EEG signals. Further, the integrity of the MRI data can be compromised by the EEG sensors. That is, the presence inside the MRI scanner of the EEG electrodes/leads with different magnetic properties from the underlying human tissues and the electromagnetic radiation emitted by the EEG system, mainly from its rapid switching digital signals, can disturb the electromagnetic fields used for imaging and compromise the quality of the MRI image scans.

Even if these design and data integrity concerns are managed, fundamental safety issues must be addressed when attempting to use EEG systems within the MRI system. The introduction of the EEG equipment into the pulsed RF fields created by the MRI scanner can also present a safety hazard, especially at high static B₀ fields, because of specific absorption rate (“SAR”) considerations. EEG leads may act as antennas, increasing the patient's exposure to the RF fields. The use of metallic electrodes and leads may cause an undesirable increase in local and whole-head SAR values, reflected in the heating of the patient's tissue. Such heating may result in bodily injury to the patient, including burns to the skin, scalp, etc.

Further still, the noise created by motion can degrade both the fMRI data and the EEG data, but in distinct ways. For example, a ballistocardiogram motion, e.g., a cardiac pulsation, within the patient and bulk movement of the patient can degrade both data sets. The noise amplitude may be approximately the same or several times to magnitude of the EEG signals, depending on the strength of the static B₀ field. However, because these motion noises may be present as a direct result of electromagnetic induction in the magnetic field, the voltage differential between the amplitude of the noise and the amplitude of the EEG signals can increase as the strength of the magnetic field increases. Ballistocardiogram noise is challenging to measure accurately, as ballistocardiogram noise is dependent on the motion of the body, the head, and the individual electrodes and may vary even between electrodes positioned close to one another. Most of all, bulk patient motion from any origin can still severely degrade the acquired information, particularly blurring the MR images rendering them inadequate for clinical evaluation.

One conventional method for removing the ballistocardiogram noise from an electrophysiological signal (e.g., EEG signals) is to subtract an average ballistocardiogram waveform created based on the electrophysiological data (i.e., an average ballistocardiogram template) from the measured electrophysiological signal. Specifically, the average ballistocardiogram template may be created by averaging every electrophysiological channel and using linear regression to create the template. However, over a predetermined time, the heart rate of the subject and/or the blood pressure of the subject may vary. Consequently, the amplitude and form of the ballistocardiogram noise signal also may change over the predetermined period. Such variations may be substantial and even occur during one or more heartbeats. As such, the average ballistocardiogram waveform may be inaccurate from one heartbeat to the next, thus introducing systematic errors in the processed electrophysiological signals. Further, because the entire electrophysiological record may be relied upon to create this average ballistocardiogram waveform, the running average ballistocardiogram waveform method may not be readily used to display continuous, real-time electrophysiological signals. Moreover, the noise associated with the movement of the subject cannot be removed from the electrophysiological signals using the average ballistocardiogram waveform method.

Thus, there is a need to acquire physiological data from a patient's brain with both high spatial and temporal resolution.

### SUMMARY

The present disclosure overcomes the aforementioned drawbacks by providing methods, systems, and arrangements for the coordinated acquisition of EEG and MRI data. More particularly, systems and methods are provided for controlling artifacts in EEG data and in MRI data, such as caused by motion or the like.

In accordance with one aspect of the disclosure, a system is provided for monitoring movement and transmitting electrical signals to or from the head of a subject during a magnetic resonance imaging (MRI) procedure performed using an MRI system. The system includes a substrate configured for placement on a head of a human subject and at least one sensor assembly configured to be supported by the support structure. The at least one sensor assembly includes a coil and an electroencephalogram (EEG) lead. The coil is fixedly secured proximate to the EEG lead and wherein the coil is electrically isolated from the EEG lead.

In accordance with another aspect of the disclosure, a system is provided for acquiring electroencephalogram (EEG) data and position data from a subject. The system includes a substrate configured to engage a head of the subject, a plurality of EEG electrodes coupled to the substrate to be positioned about the head of the subject to acquire EEG data, and a coil arranged proximate to each of the plurality of EEG electrodes to receive induced voltage caused by changes in magnetic fields proximate to each of the plurality of EEG electrodes. The system also includes a controller configured to receive an electrical signal corresponding to the induced voltage and use the electrical signal to reduce motion artifacts in the EEG data.

In accordance with yet another aspect of the disclosure, a method is provided that includes acquiring, at a plurality of different times, an induced voltage within a plurality of coils arranged about a head of a subject and positioned within a variable magnetic field. The method also includes acquiring electroencephalogram (EEG) data from a plurality of EEG sensors, wherein each EEG sensor is paired with a respective one of the plurality of coils. The method further includes determining, for each of the plurality of times, a position of each coil in the plurality of coils positioned in the variable magnetic field utilizing the induced voltage at the particular time. The method also includes using the position of each coil in the plurality of coils to correlate the EEG data with at least one of an anatomical image or a functional image of the head of the subject.

The foregoing and other aspects and advantages of the invention will appear in the following description. In the description, reference is made to the accompanying drawings that form a part hereof. There is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The foregoing and other aspects of the present disclosure will be apparent upon consideration of the following detailed description, taken in conjunction with the accompanying drawings, in which reference characters refer throughout to like parts.

FIG. 1 is a block diagram of an exemplary magnetic resonance imaging (MRI) system configured in accordance with the present disclosure.

FIG. 2 is a further block diagram of an exemplary MRI system configured in accordance with the present disclosure.

FIG. 3A is a perspective view of a carrier assembly, including a plurality of combined sensor assemblies that is fitted to a patient's head according to one example aspect of the present disclosure.

FIG. 3B is a perspective view of a mounting support of for a carrier assembly according to one example aspect of the present disclosure.

FIG. 4 is a schematic diagram of an exemplary sensor system in accordance with one example aspect of the present disclosure.

FIG. 5 is an illustrative exploded view of the components of a sensor assembly in according with one example aspect of the present disclosure.

FIG. 6A is a top view of one example of a combined sensor assembly in accordance with one example aspect of the present disclosure.

FIG. 6B is a bottom view of the example of the combined sensor assembly of FIG. 6A.

FIG. 7 is one example of a report that may be generated using the systems and methods provided by the present disclosure to identify position changes, or patient motion during acquisition of EEG data while acquiring MRI data.

### DETAILED DESCRIPTION

Generally, an exemplary aspect of the present disclosure provides methods, systems, and arrangements for acquiring EEG and MRI data in coordination. In one non-limiting example, the systems and methods provided herein may facilitate patient movement monitoring while acquiring data. Reference herein to patient signals thus includes both magnetic resonance signals detected by an MRI system during an MRI procedure and electrophysiological signals obtained using an electrophysiological patient monitoring device used in conjunction with the MRI system during the MRI procedure. For example, patient signals may include electrophysiological brain signal readings obtained using an EEG lead assembly during an MRI procedure. Signals acquired by an EEG lead assembly may be interchangeably referred to herein as electrophysiological brain signals, EEG signals, or brain waves.

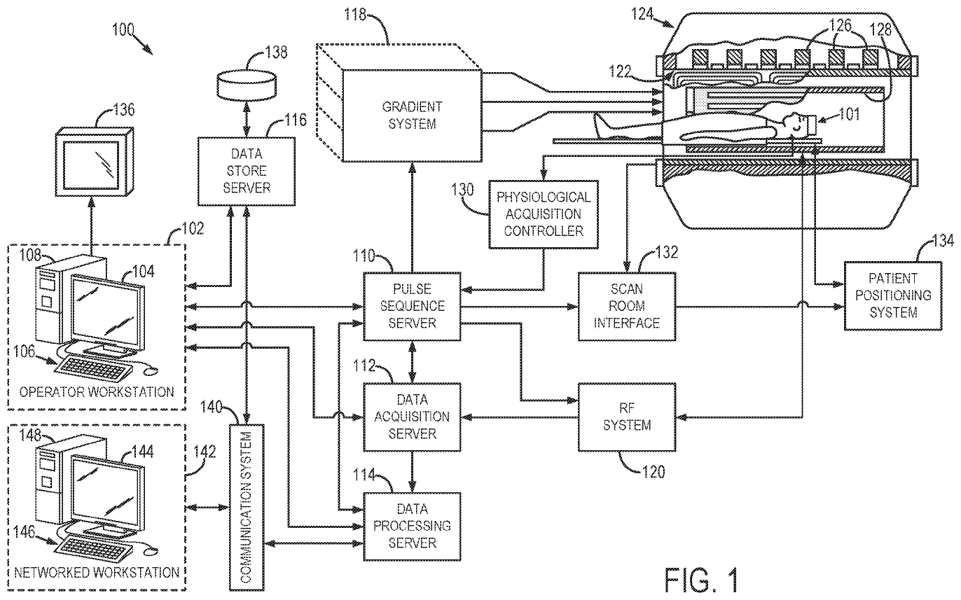

Referring now to FIG. 1, the systems and methods provided herein may be utilized with a magnetic resonance imaging (MRI) system 100, which may be configured, programmed, or used otherwise following the present disclosure, such as in coordination with an EEG system 101. The MRI system 100 includes an operator workstation 102, which will typically include a display 104, one or more input devices 106 (such as a keyboard and mouse or the like), and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to multiple servers, including a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include both proprietary or dedicated networks and open networks, such as the internet.

The pulse sequence server 110 functions in response to instructions downloaded from the operator workstation 102 to operate a gradient system 118 and a radiofrequency (RF) system 120. Gradient waveforms to perform the prescribed scan are produced and applied to the gradient system 118, which excites gradient coils in an assembly 122 to produce the magnetic field gradients Gₓ, G_{y}, G_{z} used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128 or a separate local coil such as, e.g., an optional surface coil configured to be positioned against an intended imaging target of a patient, in order to perform the prescribed magnetic resonance pulse sequence. Responsive magnetic resonance signals detected by the RF coil 128, or a separate local coil (e.g., an optional surface coil), are received by the RF system 120, where they are amplified, demodulated, filtered, and digitized under the direction of commands produced by the pulse sequence server 110. The RF system 120 includes an RF transmitter for producing a wide variety of RF pulses used in MI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils (e.g., optional surface coils) or coil arrays.

The RF system 120 also includes one or more RF receiver channels. Each RF receiver channel includes an RF preamplifier that amplifies the magnetic resonance signal received by the coil 128 (or, e.g., an optional surface coil) to which it is connected and a detector that detects and digitizes the I and Q quadrature components of the received magnetic resonance signal. The magnitude of the received magnetic resonance signal may, therefore, be determined at any sampled point by the square root of the sum of the squares of the I and Q components:

$$M = \sqrt{{I}^{2} + {Q}^{2}} ; \qquad Eqn . 1$$

and the phase of the received magnetic resonance signal may also be determined according to the following relationship:

$$φ = {tan}^{- 1} ( \frac{Q}{I} ) . \qquad Eqn . 2$$

The pulse sequence server 110 also optionally receives patient data from a physiological acquisition controller 130. By way of example, the physiological acquisition controller 130 may receive signals from a number of different sensors connected to the patient, such as electrocardiograph (ECG) signals from electrodes or respiratory signals from respiratory bellows or other respiratory monitoring devices. Such signals are typically used by the pulse sequence server 110 to synchronize, or “gate,” the performance of the scan with the subject's heartbeat or respiration.

The pulse sequence server 110 also connects to a scan room interface circuit 132 that receives signals from various sensors associated with the condition of the patient and the magnet system. Through the scan room interface circuit 132, a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage such that no data are lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, magnetic resonance data are acquired during prescans and used to calibrate the pulse sequence performed by the pulse sequence server 110. As another example, navigator signals may be acquired and used to adjust the operating parameters of the RF system 120 or the gradient system 118, or to control the view order in which k-space is sampled.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction techniques, such as iterative or back-projection reconstruction techniques; applying filters to raw k-space data or to reconstructed images; generating functional magnetic resonance images; calculating motion or flow images; and so on.

Images reconstructed by the data processing server 114 are conveyed back to the operator workstation 102. Images may be output to operator display 112 or a display 136 that is located near the magnet assembly 124 for use by the attending clinician. Batch mode images or selected real-time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. Images reconstructed by the data processing server 114 are conveyed back to the operator workstation 102. Images may be output to operator display 112 or a display 136 that is located near the magnet assembly 124 for use by the attending clinician. Batch mode images or selected real-time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

As will be described in further detail below, the MRI system 100 and the EEG system 101 may be configured to operate together in coordination. Thus, as will be described, the EEG system 101 may include a plurality of EEG leads configured to be positioned in proximity of the patient's head arranged in the bore of the MRI system 100 to acquire EEG data while acquiring MRI data. Thus, the EEG leads of the EEG system 101 are MRI compatible. Furthermore, the EEG system may be integrated with or coupled to a sensor system to form a sensor assembly capable of tracking patient position or motion and acquiring EEG and/or MRI data. By tracking the position or movement of the patient during MRI and EEG data acquisition, the systems and methods of the present disclosure are able to track physiological signals, such as brain signals, from the patient with high spatial and temporal resolution.

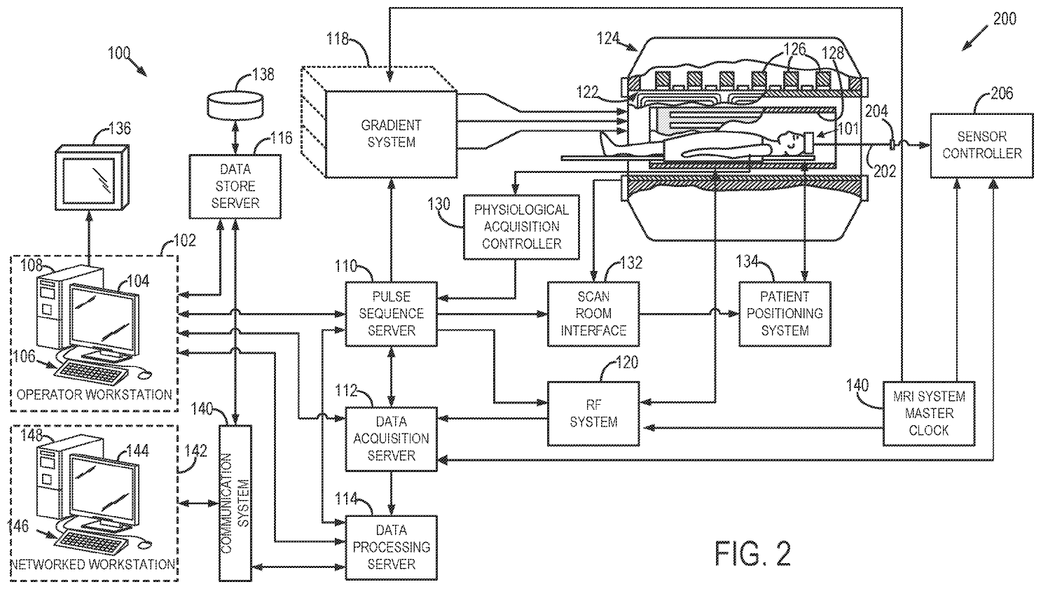

As illustrated in FIG. 2, the EEG system 101 may be coupled with a sensor system 200, which can be used with the MRI system 100. That is, referring to FIG. 2, a sensor system 200 may utilize a plurality of sensor assembly leads 202 that is coupled to the patient for acquiring physiological data and monitoring the movement of a target portion of a patient during an MRI procedure performed with the MRI system 100. In general, the sensor system 200 includes a plurality of sensor assembly leads 202 and a sensor controller 206 that receives data from the sensor assembly leads 202. Each sensor assembly leads 202 is configured to be secured relative to a target portion of a patient (i.e., a portion of the patient from which it is desired to obtain physiological signals). As will be described in more detail below, the sensor controller 206 utilizes data acquired by the sensor system 200 in coordination with the operation of the MRI system 100 to generate signals representative of the position or motion of the target portion of the patient during the MRI procedure. As such, the system can identify changes in patient position, such as caused by physiological or patient motion, and the acquired EEG and/or MRI data can be adjusted to compensate or adjust for such changes in position/motion. As such, high-temporal, high-spatial-resolution data can be provided to the clinician.

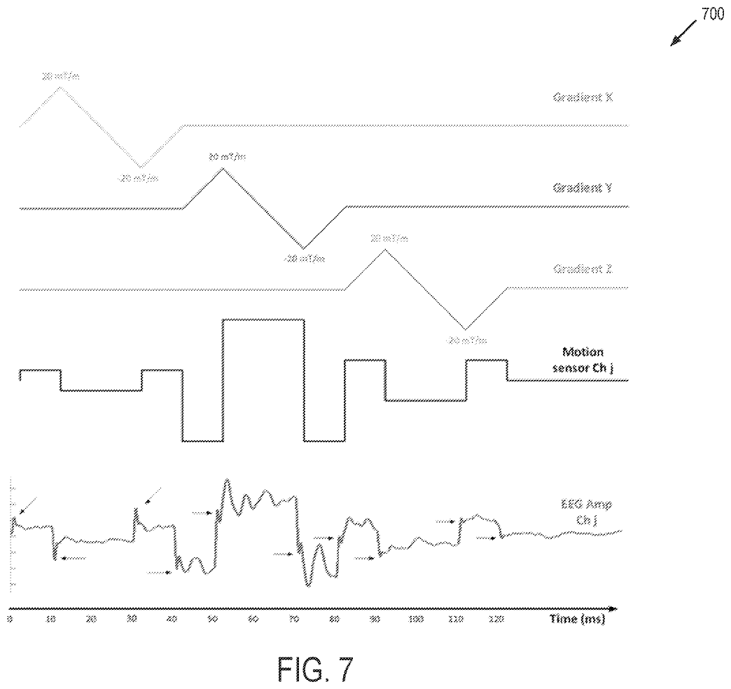

An electrical connector 204 of the sensor system 200 may be used to connect the sensor assembly leads 202 to a sensor controller 206. During the use of the sensor system 200, the electrical connector 204 allows the sensor controller 206 to obtain signals from the sensor assembly leads 202. For example, and as described in more detail below, the sensor controller 206 receives multiple signals from each of the sensor assembly leads 202, which include both physiological signals and position/motion or MRI signals. As will be described, the sensor controller 206 is able to utilize the multiple signals to combine high-temporal-resolution data (e.g., EEG data) with high-spatial-resolution data (e.g., fMRI or MRI data) to generate a report, for example, as illustrated in FIG. 7, that provides multiple sources of physiological and/or anatomical information with improved resolution and reduced artifacts compared to traditional systems that do not offer sensor assembly leads 202 that can deliver multiple, distinct data signals simultaneously. As illustrated, the sensor controller 206 may be connected to a MRI system clock 140. That is, the MRI system 100 includes a master clock 140 that connects to the various parts of the MRI system 100 that generate signals, such as the RF system 120 and the gradient system 118, as just one example. In this way, though the MRI system 100 operates a clock frequency that is generally appreciably greater than the EEG system 101, such as 10 MHz versus less than 50 Hz, the master clock 140 provides a resource through which to synchronize the MRI data with the data from the EEG system 101. In this way, the noise can be systematically sampled so that, for at each repetition time (TR) of the MR pulse sequence, the noise is sampled in the same fashion and appears to be the same. That is, as will be described, the EEG system 101 can include a combined EEG electrode and motion coil that, together, allow motion or position information to be identified, such as illustrated in the report 700 illustrated in FIG. 7. According to various aspects of the present disclosure, the sensor controller 206 can also be connected to the communication system 140 of the MRI system 100 or the data acquisition server 112 or other servers 110, 114, 116 of the MRI system 100.

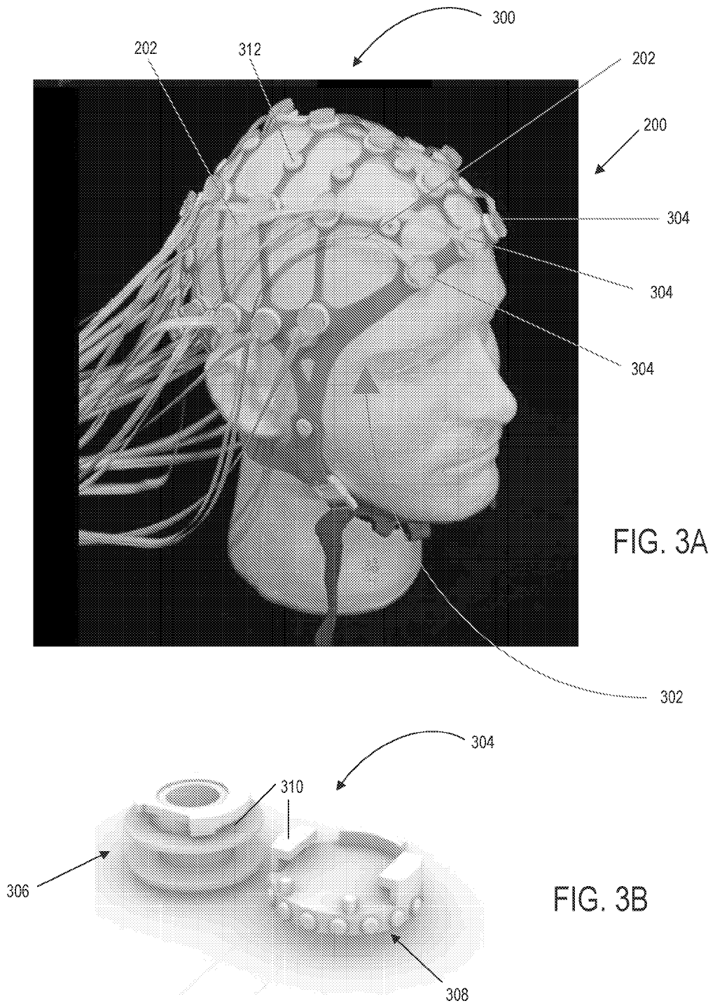

Referring to FIG. 3A, one non-limiting example of the sensor system 200 is illustrated. The sensor system 200 can include a carrier assembly 300 that is designed to position a plurality of sensor assembly leads (not shown in FIGS. 3A and 3B) about a subject's head via a mounting system 304 to acquire patient signals from multiple patient sites during an MRI procedure is shown according to one example configuration. The carrier assembly 300 generally includes a support structure 302 that forms a contoured helmet or flexible cap. Alternatively, in some designs, the carrier assembly 300 may instead, or additionally, include a plurality of sensor assemblies 200 that independently attach to the patient.

As will be described, each sensor assembly lead is capable of acquiring physiological data and movement data. The carrier assembly 300 advantageously allows for the precise monitoring of movement at each patient location from which signals are obtained. By allowing for such localized and individualized motion detection, the carrier assembly 300, as will be described, overcomes the shortcomings of traditional EEG systems.

The support structure 302 of the carrier assembly 300 facilitates the attachment of the plurality of sensor system leads via the mounting system 304 to a patient. The support structure 302 is configured to be attached to (e.g., worn) by a patient during an MRI procedure. The support structure 302 is designed to be invisible to MRI. For example, the support structure 302 may be constructed from silicone or other MRI-invisible/MRI-compatible material and/or a material that is compatible with other imaging modalities, such as computed tomography (CT) imaging. The configuration of the support structure 302 is adapted to the patient's particular anatomy from which signals are to be obtained. For example, as illustrated by FIG. 3A, in configurations in which the carrier assembly 300 is utilized to obtain EEG signals during an MRI procedure, the support structure 302 is configured as a cap that is fitted to the head of a patient.

Referring to FIGS. 3A and 3B, the sensor assembly lead (now shown in FIGS. 3A and 3B) may terminate in the mounting system 304 configured to engage the support structure 302. In particular, the mounting system 304 may be formed of a sensor mount 306 and a locking mount 308. That is, the sensor mount 306 is configured to secure a sensor (which, as will be described, may be a combined EEG and MRI sensor) against the patient when the support structure 302 of the carrier assembly 300 is positioned on the patient. The locking mount 308 mates with sensor mount 306. For example, a set of locking keys 310 may allow the locking mount 308 and the sensor mount 306 to be arranged on opposing sides of the support structure 302 and lock the support structure 302. As illustrated in FIG. 3A, a plurality of locations for arranging the sensor systems leads may be provided, and not all need to be used at any given time. For example, as shown in FIG. 3A, empty locations 312 may be provided. That is, each mounting system 304 is configured to releasably secure a combined sensor assembly relative to the support structure 302.

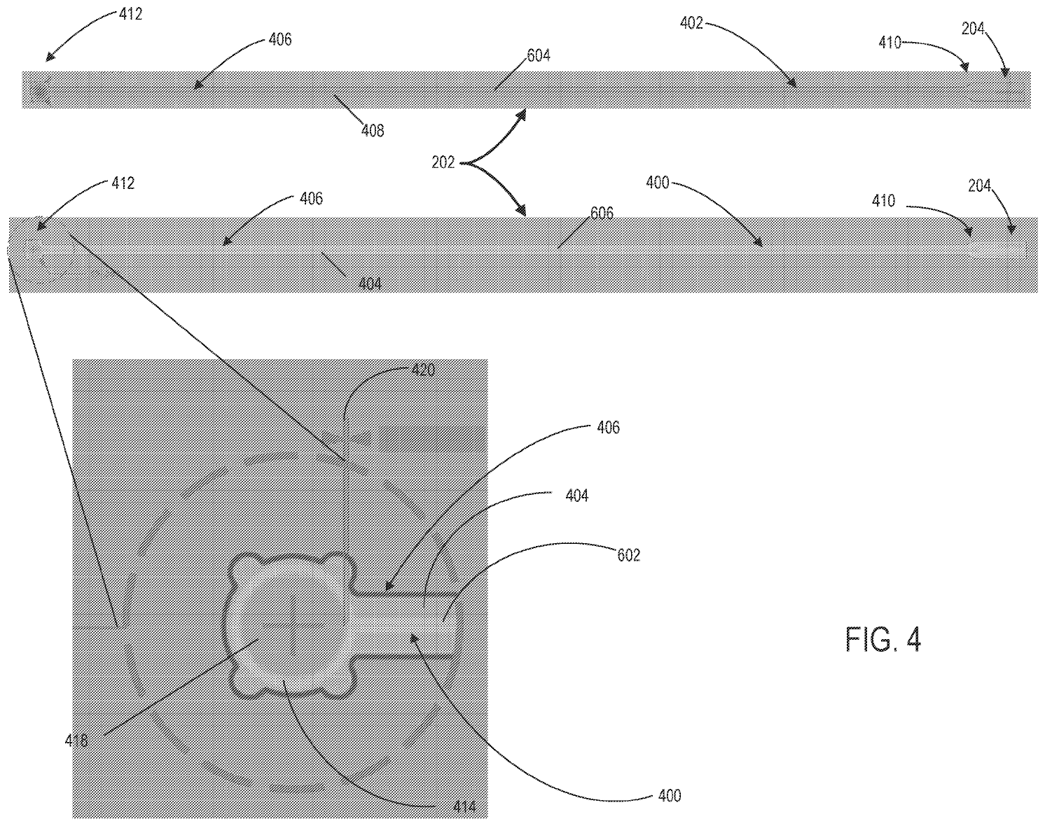

Referring to FIG. 4, in one non-limiting configuration, the sensor assembly lead 202 may include a first circuit 400 and a second circuit 402. In one non-limiting example, the first circuit 400 may extend along a first or top surface 404 of a substrate 406, and the second circuit may extend along a second or bottom surface 408, such that each circuit 400, 402 extends between a proximal end 410 and a distal end 412. By having two circuits 400, 402, the sensor assembly leads 202 is able to acquire two distinct signals. Specifically, the first circuit 400 includes an electrical loop or coil 414 that is located at the distal end 412 and includes traces 416 extending to the proximal end 410. As will be described, the loop/coil 414 is configured to acquire one-time information, such as MR or position data. The second circuit 402 includes an electrical contact 418 that is located the proximal end 410. As will be described, the electrical contact 418 can be configured to acquire physiological data, such as EEG data. In the non-limiting example shown in FIG. 4, the electrical contact 418 can be arranged, as illustrated, to be circumscribed by the loop/coil 414. In one non-limiting example, a spacing therebetween 420 may be selected to make the distal end 412 of the sensor assembly lead 202 be highly compact, such as can be arranged in the mounting system 304 of FIGS. 3A and 3B and be arranged at a plurality of locations about the patient, despite having two distinct electrical components. In one non-limiting example, the spacing 420 may be 0.010 inches.

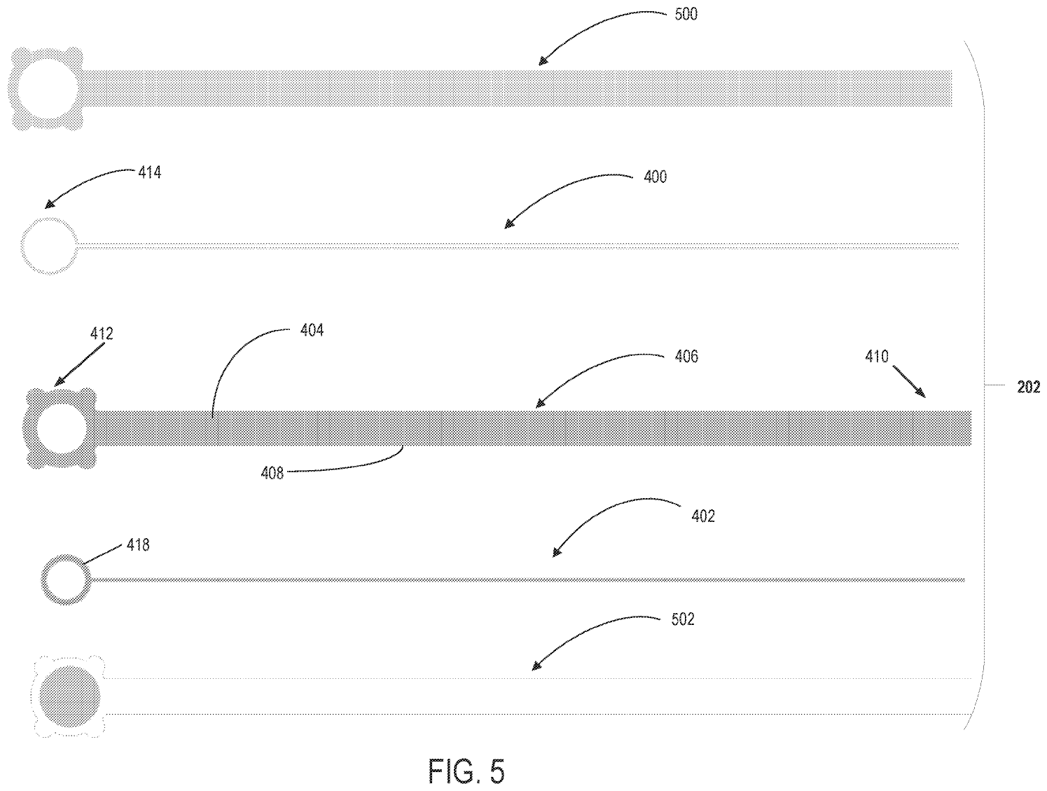

As shown in the illustrative exploded view of FIG. 5, the sensor assembly lead 202 can be formed in multiple layers. As described, sensor assembly lead 202 includes a substrate 406 that can receive the first circuit 400, including the loop/coil 414, and the second circuit 402, including the electrical contact 418. In one non-limiting example, the first circuit 400, including the loop/coil 414 may be formed on the first or upper surface 404 of the substrate 406, and the second circuit 402, including the electrical contact 418 may be formed on the second or lower surface 408 of the substrate 406.

The substrate 406 provides structural support for the circuits 400, 402. Substrate 406 can be a generally flexible substrate formed of one or more layers made of one or more non-conductive materials. One or more optional protective layers 500, 502 may be included. The optional protective layer(s) 500, 502 may be secured over either or both of the circuits 400, 402. The optional protective layers 500, 502 may include one or more layers of materials that are selected to provide the circuit 400, 402, with different properties and/or forms of protection. For example, one optional protective layer 500, 502 may be used to provide electrical insulation, waterproofing, chemical insulation, and/or protection against physical damage (e.g., scratching, delamination from the base layer, etc.).

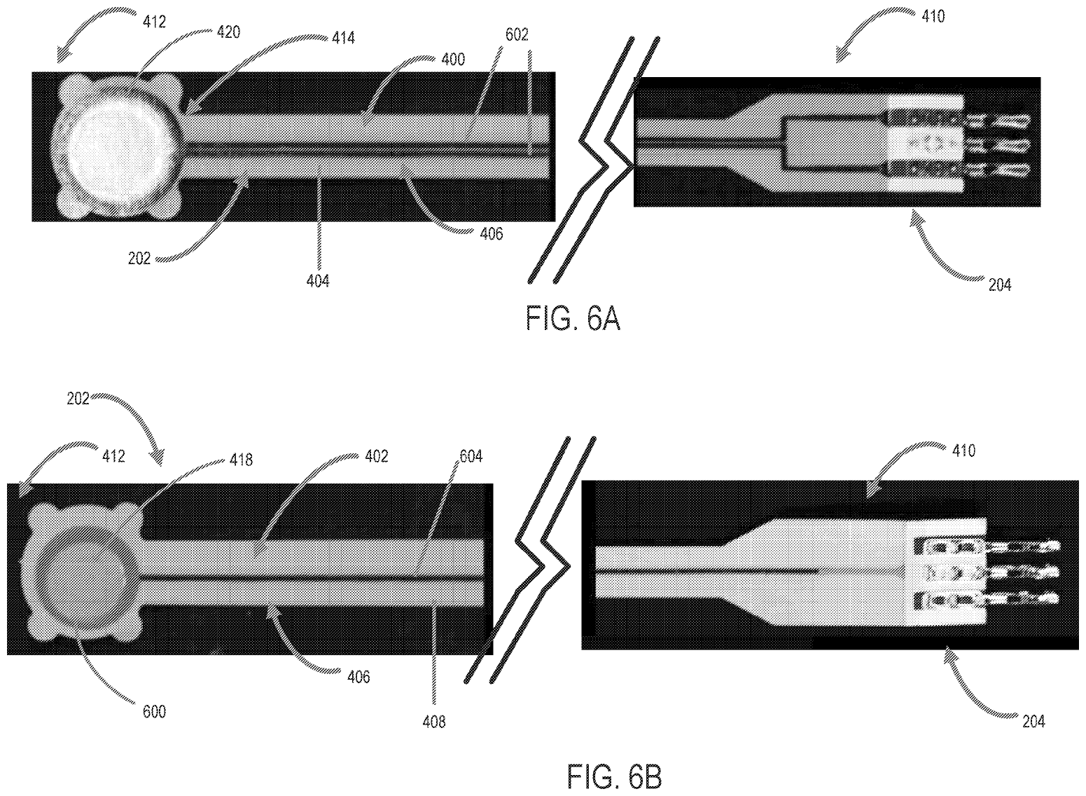

Referring to FIGS. 6A and 6B, one non-limiting example of the sensor assembly lead 202 is shown from a top view (FIG. 6A) and a bottom view (FIG. 6B). As illustrated, the loop/coil circuit 414 can be seen on the top view (FIG. 6A), and the electrical contact 418 can be seen on the bottom view (FIG. 6B) and the top view (FIG. 6A). In this way, when the bottom view is arranged against the patient, the electrical contact 418 is coupled to the patient. On the other hand, the loop/coil circuit 414 is displaced from the patient by substrate 406. As will be described, this highly functional design allows the electrical contact 418 to engage the patient to acquire physiological information such as EEG signals, while the loop/coil circuit 414 can be separated by the substrate 406 and still acquire motion or position signals, such as via MRI signals. However, as illustrated and described, the loop/coil circuit 414 and electrical contact 418 are proximate to each other. This can be achieved, for example, using a passage 600 formed in the substrate 406 through which the electrical contact 418 extends.

To keep the two circuits 400, 402 electrical distinct/isolated, electrical connections, such as traces 602, 604, extending from the distal end 412 of the substrate 406 to the proximate end 410 may be arranged on opposing sides of the substrate. For example, as illustrated in FIG. 6A, the traces 602 extending to/from the loop/coil circuit 414 extend along the first side (top) of the substrate 406, whereas the trace 604 extending from the electrical contact 418 extend along a second side (bottom) of the substrate 406.

According to some configurations, the sensor assembly lead 202 can be constructed using polymer thick-film technology. In some such configurations, the first circuit 400 can be formed from a conductive Polymer Thick Film (PTF) that is deposited (e.g., via printing) along the upper surface 404 of the substrate or base layer 406. The conductive ink(s) used to form the loop/coil circuit 414 and traces 602 extending from the loop/coil circuit 414 may include one or more conductive non-ferrous materials such as metals (e.g., copper or silver) and/or metal ions (e.g., silver chloride), filler-impregnated polymers (e.g., polymers mixed with conductive fillers such as graphene, conductive nanotubes, metal particles), or any conductive ink capable of providing conductivity at levels suitable for detecting signals acquired by the loop/coil circuit 414 during use of the sensor system 200.

In the non-limiting example illustrated in FIGS. 6A and 6B, the thick-film technology may also be used to form the optional protective layer 500, 502 described with respect to FIG. 5. Alternatively, or additionally, the optional protective layer 500, 502 may be provided as a discrete component that is attached (e.g., using adhesive).

In some non-limiting examples, the loop/coil circuit 414 and traces 600 or the further trace 602 may be formed using a fabrication process including a thin-film deposition step and a track-patterning step. During the thin-film deposition step, a very thin layer (i.e., a layer having a thickness of less than about 100 nm, and preferably less than about 50 nm) of the conductor is deposited along the upper surface 404 of the substrate 406. The circuits 400, 402 may comprise any number of non-ferrous conductive materials that are capable of being deposited as a thin film (i.e., are capable of being applied to the substrate 406. In some configurations, the substrate 406 may have a thickness of less than about 1 μm, and preferably less than about 100 nm, and more preferably less than about 50 nm.). Non-limiting examples of thin-film fabrication methods via which the track-forming material may be deposited along the substrate 406 include metallization, chemical or physical vapor deposition, plating, chemical solution deposition, spin coating, chemical vapor deposition (“CVD”), sputtering, or other suitable processes that allow for the deposition of the track-forming material with a thickness of less than about 1 μm, and preferably less than about 100 nm, and more preferably less than about 50 nm.

During the track-patterning step, portions of the material deposited on the substrate 406 to form the circuit 400, 402 during the thin-film deposition step can be selectively removed to create the loop/coil circuit 414 and/or traces 600, 602 to have the desired width, length, and track pattern. Non-limiting examples of methods that may be used to remove the deposited track-forming material selectively include lithography, etching, trimming, lift-off, or other suitable processes. Alternatively, in some configurations, the track-patterning step may involve the application of a mask or stencil having the desired track pattern onto the upper surface 404 of the substrate 406. Once the desired track pattern has been outlined along the upper surface 404 of the substrate 406, the track-forming material is deposited along the upper surface 404 of the substrate 406 (and mask/stencil applied along a portion thereof) during a thin-film deposition step to form the conductive coil/loop 408 and traces 600, 602, following which the mask/stencil is optionally removed. As shown in FIGS. 6A and 6B, the proximal end 410 includes the electrical connector 204 to be coupled to the controller 206 of FIG. 2.

According to yet other aspects, the sensor assembly 200 may be constructed utilizing any number of other fabrication techniques. For example, the sensor assembly 200 may include a discretely provided conductor formed from a non-ferrous conductive structure (e.g., wire, filament, etc.) that is secured (e.g., by adhesion, embedding, encapsulation, etc.) relative to the upper surface 404 of the substrate 406 to form the sensor assembly 200.

Referring to FIGS. 4-7, by creating a sensor assembly lead 202 with two distinct electrical circuits 400, 402, one forming the loop or coil 414 and the other forming the electrical contact 418, the sensor assembly lead 202 can function to acquire two distinct forms of data. In particular, the electrical contact 418 is configured to acquire physiological data from the patient. For example, the physiological data can be EEG data. Additionally, the loop or coil 414 can be configured to acquire position or other data. For example, because the sensor assembly lead 202 is configured to be positioned in an MRI system, the loop or coil 414 can be configured to operate as an MRI coil to acquire MRI data. As will be described, the MRI data can be used to monitor the position or motion of the patient. In this way, the MRI data acquired by the loop/coil 408 can be used to register the EEG data acquired by the contact 418 with, for example, fMRI data acquired by the MRI system, such that the EEG and fMRI data are registered even in the face of motion (physiological motion or bulk patient motion). This is possible because the loop/coil 414 is coupled to the patient and the contact 418. Thus, the data acquired by the loop/coil 414 is an absolute reference with respect to the patient and the contact 418. As such, EEG and fMRI data can be combined to provide the high spatial resolution of the MRI data and the high temporal resolution of the EEG data to the clinician without the drawbacks of misregistration and/or other artifacts. That is, the data from the loop/coil 414 can be used to identify and compensate for motion readily (physiological or bulk patient motion) and then compensate for the motion when combining the fMRI data with the EEG data.

Additionally or alternatively, the loop/coil 414 may be used to acquire fMRI or other data directly from the patient. Again, because the loop/coil 414 is coupled to the patient and the contact 418, the data acquired by the loop/coil 414 is necessarily registered to the patient and to the contact 418. As such, EEG and fMRI data can be combined to provide the high spatial resolution of the MRI data and the high temporal resolution of the EEG data to the clinician without the drawbacks of misregistration and/or other artifacts. Furthermore, because the sensor system 200 presents an array of sensor assembly leads 202, the MR data acquired by the loop/coil 414 provides substantial spatial resolution with substantial SNR, which is far superior to even birdcage or similar MR coils used to acquire fMRI data.

Referring again to FIGS. 2-5, as discussed above, patient movement during an MRI procedure may introduce noise into patient signals obtained during an MRI procedure. Such movements may include head movements by the subject, swallowing by the subject, movement during respiration, noise associated with a blood flow motion within the subject, and noise associated with a ballistocardiac motion (e.g., a cardiac pulsation) within the subject, etc. The motion artifacts in these obtained patient signals may decrease the accuracy of the final output readings generated by the MRI procedure.

As described above, spatial encoding relies on successively applying magnetic field gradients in an MRI system. A slice selection gradient is used to select the anatomical volume of interest. Within this volume, the position of each point is encoded vertically and horizontally by applying a phase encoding gradient, and a frequency-encoding gradient. The different gradients used to perform spatial localization have identical properties but are applied at distinct moments and in different directions (i.e., x, y, and z). Gradient equivalence in the three directions of space means that slices can be selected on any spatial plane. During MRI spatial localization gradient switching, because of Farday's law of induction, voltages are induced in the loops, such as loop/coil circuit 414, as the magnetic flux changes over time, as given by:

$${V}_{k} ( t ) = {w}_{x , k} \frac{\partial {φ}_{x , k} ( t )}{\partial t} + {w}_{y , k} \frac{\partial {φ}_{y , k} ( t )}{\partial t} + {w}_{z , k} \frac{\partial {φ}_{z . k} ( t )}{\partial t} + {ε}_{k} ( t ) ; \qquad Eqn . ( 3 )$$

where φ_{x,k} is the flux over the k-th loop for the x-directional gradient, φ_{y,k} and φ_{z,k} refer to fluxes due to the y and z directional gradients. Whereas, w_{x,k}, w_{y,k} and w_{z,k} are weights function of the loop position and orientation, and εₖ (t) is the noise all referred to the k-th loop. Thus, the above-described system provides a system that includes a loop/coil circuit 414 that acquires voltage information that can then be used to determine the position or any change in position of each of the loops/coils within the MRI bore.

Thus, monitoring and detecting patient movement during an MRI procedure may advantageously allow motion-related artifacts to be filtered from obtained patient signals to improve the quality of the final output readings generated by the MRI procedure. Additionally or alternatively, such information may be used to register the information acquired with the MRI system with other information, such as EEG data. However, as will be appreciated, the accuracy of the final output readings will vary based on the accuracy of the obtained patient movement measurements. In particular, patient movement measurements that precisely measure actual patient movement are more likely to provide final output readings that more accurately reflect the true (i.e., actual) patient condition/signal being monitored.

However, the ability to filter out motion artifacts to improve the quality of final output readings or improve data registration requires more than just highly accurate movement measurements. Rather, the obtained movement measurements are desirably also as specific as possible to the portion of the patient from which the patient signals were obtained. Notably, highly precise movement measurements may have little (e.g., no) effect on improving the accuracy of a final output reading if the movement measurements do not reflect patient movement at the site from which the patient signals were detected (i.e., if the movement measurements reflect the movement of a different portion of the patient). Thus, obtaining movement measurements from highly localized and specific portions of the patient (i.e., movement measurement reflective of movement in small, specific target areas) that correspond to the specific (e.g., exact) location from which patient signals are obtained may enhance the final output readings.

As described above, the systems and methods provided herein provide highly-sensitive, highly-localized positional information. In some embodiments, in addition to being utilized as a motion sensor, the above-described systems and methods may optionally also be utilized as a local coil of the MRI system. Accordingly, in some configurations, the above-described systems and methods may be configured to interface with the components of the MRI system 100 (e.g., the RF system 120) to process magnetic resonance signals received while acting in its capacity as a local coil of the MRI system 100.

According to various aspects of the present disclosure, the above-described systems and methods may include one or more filtering programs including one or more filtering routines to utilize the motion measurement data acquired by the loop/coil circuit 414 to remove motion-induced noise artifacts from the EEG signals received by the electrical contact 418. For example, according to one non-limiting example, the system may include a filter program in which an EEG signal y(t) (i.e., the EEG signal obtained from the electrical contact 418) is modeled as the sum of a “true” underlying EEG signal s(t) and a signal n(t), known to include motion and ballistocardiogram components:

$$y ( t ) = s ( t ) + n ( t ) . \qquad Eqn . 4$$

The relationship between the noise signal n(t) and motion measurement signals m(t) (i.e., the motion signal obtained from the loop/coil circuit 414) is modeled linearly using a time-varying, finite impulse response (FIR) kernel wₜ(k) with the equation:

$$n ( t ) = {\sum }_{k = 0}^{N - 1} {w}_{t} ( k ) m ( t - k ) ; \qquad Eqn . 5$$

where Nis the order of the FIR kernel. The system can use an adaptive filtering algorithm to produce an estimate of the FIR kernel wₜ(k), which is in turn used to estimate the noise signal n(t):

$$\hat{n} ( t ) = {\sum }_{k = 0}^{N - 1} {\hat{w}}_{t} ( k ) m ( t - k ) . \qquad Eqn . 6$$

The estimated noise signal is then subtracted from the recorded signal y(t) to reveal the underlying EEG signal s(t):

$$\hat{s} ( t ) = y ( t ) - \hat{n} ( t ) . \qquad Eqn . 7$$

Additional information related to adaptive filtering algorithms which the combined system may use is detailed in the paper “MOTION AND BALLISTOCARDIOGRAM ARTIFACT REMOVAL FOR INTERLEAVED RECORDING OF EEG AND EPS DURING MRI,” written by Giorgio Bonmassar et al., and published in NeuroImage 16, pgs. 1127-1141 (2002), the entirety of which is incorporated herein by reference.

As will be appreciated, the construction of the substrate 406 (i.e., the ability of the substrate to be secured relative to a patient) and the configuration of the loop/coil circuit of the sensor assembly 200 may allow the sensor assembly 200 to act as a surface coil (i.e., a local coil) of the RF system 120 of the MRI system 100. Accordingly, in some configurations, the combined sensor assembly may be utilized to monitor patient movement, obtain patient signals (e.g., EEG signal) from the lead assembly, and receive magnetic resonance signals during an MRI procedure.

As discussed above, the ability to monitor patient movement at the precise location from which patient signals (e.g., received magnetic resonance signals) are being obtained may advantageously be utilized to remove motion-related noise artifacts from received patient signals. Accordingly, by combining the ability of the sensor assembly 200 to receive magnetic resonance signals from a target portion of a patient during an MRI procedure (i.e., utilizing the sensor assembly 200 as a surface coil) with the ability of the sensor assembly 200 to obtain precise movement measurements from the target portion of the patient from which the magnetic resonance signal is received, the sensor assembly 200 may advantageously enhance the quality of the final output readings (e.g., MRI images) generated during the MRI procedure.

Although the patient signal acquisition assembly is shown and described as comprising an EEG lead assembly, as will be appreciated, in other aspects, the patient signal acquisition assembly may comprise any variety of other devices that are configured to obtain/detect position-sensitive signals from a patient during an MRI procedure.

The present invention has been described in terms of one or more preferred embodiments, and it should be appreciated that many equivalents, alternatives, variations, and modifications, aside from those expressly stated, are possible and within the scope of the invention.

As used in the claims, the phrase “at least one of A, B, and C” or “at least one of A, B, or C” means at least one of A, at least one of B, and/or at least one of C, or any one of A, B, or C or combination of A, B, or C. A, B, and C are elements of a list, and A, B, and C may be anything contained in the Specification.

What is claimed is:

1. A system for monitoring movement and transmitting electrical signals to or from the head of a subject during a magnetic resonance imaging (MRI) procedure performed using an MRI system, the system comprising:
a substrate configured for placement on a head of a human subject; and
at least one sensor assembly configured to be supported by the support structure, the at least one sensor assembly comprising:
a coil;
an electroencephalogram (EEG) lead; and
wherein the coil is fixedly secured proximate to the EEG lead and wherein the coil is electrically isolated from the EEG lead.

2. The system of claim 1, further comprising a controller configured to receive motion data from the coil and EEG data from the EEG lead

3. The system of claim 2, wherein the controller is configured to utilize the motion data to correct errors in at least one of the EEG data or MRI data acquired by the MRI system.

4. The system of claim 3, wherein the controller is configured to use the motion data to remove motion artifacts from one of the EEG data or the MRI data or localize the position or motion of each electrode or the head of the subject.

5. The system of claim 3, wherein the controller is configured to use the motion data to register the EEG data and the MRI data.

6. The system of claim 1, wherein the EEG lead comprises:
an electrode that extends through a surface of the substrate; and
a conductive trace that extends along the surface of the substrate.

7. The system of claim 1, wherein the coil is mounted on a first side of the substrate, and the EEG lead is mounted on a second side of the substrate.

8. The system of claim 1, further comprising a controller configured to:
detect, at a plurality of different times, an induced voltage within the coil;
obtain information about a gradient field applied by the MRI system at each of the plurality of different times; and
calculate, for each of the plurality of times, a position of the coil utilizing the induced voltage at the particular time and gradient information obtained from the MRI system for the particular time.

9. The system of claim 8, wherein the controller is further configured to:
generate a movement or position measurement signal using the position of the coil at the plurality of different times;
obtain an EEG signal from the EEG lead; and
filter the movement measurement signal from the EEG signal.

10. The system of claim 1, wherein the at least one sensor assembly includes a plurality of sensor assemblies configured to be positioned on the subject during the MRI procedure.

11. The system of claim 10, wherein the plurality of sensor assemblies includes at least 32 sensor assemblies.

12. The system of claim 10, wherein each of the plurality of sensor assemblies forms an EEG helmet and co-registered MRI coils.

13. A system for acquiring electroencephalogram (EEG) data and position data from a subject, the system comprising:
a substrate configured to engage a head of the subject;
a plurality of EEG electrodes coupled to the substrate to be positioned about the head of the subject to acquire EEG data;
a coil arranged proximate to each of the plurality of EEG electrodes to receive induced voltage caused by changes in magnetic fields proximate to each of the plurality of EEG electrodes; and
a controller configured to receive an electrical signal corresponding to the induced voltage and use the electrical signal to reduce motion artifacts in the EEG data.

14. The system of claim 13, wherein the controller is configured to communicate with a magnetic resonance imaging (MI) system to determine a gradient field applied by the MRI system proximate to each of the plurality of EEG electrodes.

15. The system of claim 14, wherein the controller is configured to register the EEG data with MRI data acquired by the MRI system or register the MRI data with the EEG data.

16. The system of claim 14, wherein the controller is configured to filter movement determined from the induced voltage from the EEG data or MRI data acquired by the MRI system.

17. The system of claim 14, wherein the substrate forms a cap configured to be attached to the subject to position the EEG electrodes and respective coils about the head of the subject

18. The system of claim 14, wherein each coil surrounds a respective one of the plurality of EEG electrodes.

19. A method comprising:
acquiring, at a plurality of different times, an induced voltage within a plurality of coils arranged about a head of a subject and positioned within a variable magnetic field;
acquiring electroencephalogram (EEG) data from a plurality of EEG sensors, wherein each EEG sensor is paired with a respective one of the plurality of coils;
determining, for each of the plurality of times, a position of each coil in the plurality of coils positioned in the variable magnetic field utilizing the induced voltage at the particular time; and
using the position of each coil in the plurality of coils to correlate the EEG data with at least one of an anatomical image or a functional image of the head of the subject.

20. The method of claim 19, wherein correlating the EEG data with the at least one of the anatomical image or a functional image of the head of the subject includes compensating for motion of the subject when acquiring the EEG data.
